(19) United States
(12) Patent Application Publication
(10) Pub. No.: US 2026/0363908 A1
(43) Pub. Date: Nov. 19, 2026

(54) FLEXIBLE SLEEVE FOR THE TIP OF AN OPHTHALMIC SURGICAL INSTRUMENT

(71) Applicant: Ademar Jaime CARNEIRO, Piracaia (BR)

(72) Inventors: Henrique MONTEIRO BALARIN SILVA, Limeira (BR); André RAPOSO MONSANTO, Campinas (BR); Rafaello SALA PASQUINELLI, Pouso Alegre (BR); Fabrício TENO CASTILHO BRAGA, Araçatuba (BR); Bruno TENO CASTILHO BRAGA, São José do Rio Preto (BR); Márcio PRESTES LENHARO, Limeira (BR); Ademar Jaime CARNEIRO, Piracaia (BR)

(73) Assignee: Ademar Jaime CARNEIRO, Piracaia, SP (BR)

(21) Appl. No.: 18/995,904

(22) PCT Filed: Jun. 10, 2024
(86) PCT No.: PCT/BR2024/050243

§ 371 Date: Jan. 17, 2025

(30) Foreign Application Priority Data
Jul. 13, 2023 (BR) 1020230141382

(51) Int. Cl. A61F 9/007

(52) U.S. Cl. CPC A61F 9/00736

(57) ABSTRACT

The present invention relates to a protective silicone sleeve fitted over the tip of one of the two instruments used in phacoemulsification surgeries. The said silicone sleeve is designed with specific technical characteristics to fully cover the metallic tip of the phaco handpiece, one of the aforementioned instruments, so that said phaco handpiece can perform the same function as the other instrument used in phacoemulsification surgeries, namely the irrigation and aspiration handpiece, thereby eliminating the need for its use.

### TECHNICAL FIELD

The present invention is included in the technical field relating to surgical instruments, and more specifically it concerns a protective silicone sleeve fitted over the tip of one of the two instruments used in phacoemulsification surgeries, said silicone sleeve designed with specific technical characteristics to fully cover the metallic tip of the phaco handpiece, one of the aforementioned instruments, so that said phaco handpiece can perform the same function as the other instrument used in phacoemulsification surgeries, namely the irrigation and aspiration handpiece, thereby eliminating the need for its use.

### BACKGROUND OF THE INVENTION

As professionals in the field of ophthalmology know, the human eye has a lens, appropriately named Crystalline Lens, which allows light to form a focus on the macula in the retina, a sensory membrane that lines the eye internally. Due to several reasons ranging from physical trauma, age, or ophthalmological diseases, said crystalline lens can gradually become opaque or cloudy and, thus, negatively affect an individual's ability to see. This clouding of the crystalline lens is known as a cataract, an eye disease that specifically affects the crystalline lens of an eye, both in humans and in other mammals such as domestic dogs and cats.

There are several surgical procedures for treating cataracts that use distinct types of instruments and devices, in particular cutting instruments to incise the crystalline lens affected by the cataract, in order to replace it with an artificial lens. A very common procedure for cataract treatment is called phacoemulsification, a type of intraocular surgery, a surgical technique that uses specific instruments and devices to insert the tip of one of the instruments into the eyeball and remove the crystalline lens of the eye with the cataract, replacing it with an artificial intraocular lens (IOL). More specifically, one of the instruments involved in phacoemulsification is called a phaco handpiece or phaco cannula, which is connected to an ultrasonic device, in which the handpiece makes an incision in the limbus region of the eye, the region where a colored part meets a white part, wherein the incision can also be made in the cornea of the eye. The inner tube, known as the needle, is subjected to ultrasonic vibration through said ultrasonic device, so that its vibrating tip member of the phaco handpiece operates to emulsify the hard nuclear material of the cataractous lens.

Once inserted into the eyeball, the vibrating inner tube of the phaco handpiece also functions as an aspirator, so that the emulsified material from the cataractous lens can be sucked out of the eye. The outer tube functions as an irrigator and allows a saline fluid to enter the eye. Said fluid particularly prevents the cornea from collapsing when the lens material is emulsified and sucked out, and also aids in sucking the cataractous lens material out of the eye.

In conjunction with the phaco handpiece, the other instrument involved in phacoemulsification, referred to as the irrigation and aspiration handpiece, performs more precise and delicate movements than the phaco handpiece, and aspirates the smaller internal parts of the cataractous lens that were not aspirated by the phaco handpiece.

Both instruments, in this case the phaco handpiece and the irrigation and aspiration handpiece, have a metallic tip that is threaded, in which said tip is covered by a structure called a sleeve, which can be made of flexible silicone, and is fitted over the metallic tips of each surgical instrument. In particular, the silicone sleeve provides better sealing of the cut and more stable maintenance of the eye anterior chamber during surgery, something that contributes to better healing of the patient. Furthermore, the silicone sleeve is used in the phaco handpiece to direct the flow of Balanced Saline Solution (BSS) irrigation, in order to maintain the stability of the anterior chamber, and also prevents possible burns of the incision made by the surgical instruments by cooling their metallic tip. Currently, the silicone sleeve usually has dimensions for corneal incisions of 2.75 mm and 2.2 mm.

Furthermore, since the irrigation and aspiration handpiece has a thinner tip compared to the tip of the phaco handpiece, a well-constructed main incision is crucial to minimize the efflux of fluid from the patient's eye.

In this sense, it is a concern of the state of the art to present solutions that allow a patient to recover better from the phacoemulsification procedure, and that prevent excessive efflux from the patient's eye during the procedure, otherwise there may be serious consequences for the patient, such as total loss of vision or even of the eye itself.

Examples of solutions for better recovery and greater efficiency of the procedure in question are exemplified as disclosed in document U.S. Pat. No. 5,725,495, which presents a phacoemulsification instrument that reduces the leakage of infusion fluid by providing the silicone sleeve with an embedded reinforcing member. In addition, the covering has only a single infusion hole to provide a better flow of infusion fluid without causing turbulence in the eye. Furthermore, the present invention provides a variable opening suction valve or tube to regulate the suction rate. In addition, the present invention includes a single aspiration hole in the tip to amplify the flow arriving to the tip. To aid in cataract removal, the tip can be provided with a variety of different barrier and deflector configurations. A handpiece includes a variable-capacity reservoir in the infusion line to account for surges in the aspiration line. The tip includes steps, angles, and barriers to focus the ultrasonic energy to aid in emulsifying the nuclear tissue and to help pushing the emulsified material in the direction of the aspiration flow. A compressible covering is rotatably attached to the handpiece to allow the handpiece and tip to be manipulated to a limited extent in the axial direction and rotated freely about the longitudinal axis while the covering remains stationary relative to the cornea.

Another example is disclosed by document U.S. Pat. No. 6,013,046, which presents a tip protected by a covering, to be inserted through an incision for maintaining the suction and irrigation flows includes a portion of soft sleeve around a small diameter tip, in a spacing sufficient to provide a flow gap for the irrigation fluid while the suction fluid flows in the opposite direction through the center of the tip. A radially rigid tubular element within a proximal portion of the sleeve maintains a constant flow gap, allowing the edges of a small incision into which the tip is inserted to compress the soft sleeve without reducing the gap. At the distal end, however, the cover can compress in response to tissue resistance during insertion, minimizing the risks of tearing the tissue or enlarging the incision.

However, these solutions still require the surgeon to use the two instruments, the phaco handpiece and the irrigation and aspiration handpiece together to perform the phacoemulsification procedure on a patient, which generates additional costs due to the surgeon's need to have both instruments, as well as silicone sleeves for the metallic tips of each instrument. Furthermore, due to the fact that two instruments are required, both instruments must be prepared, which basically includes their sterilization and calibration of the ultrasound machine for each specific instrument.

Furthermore, the surgeon or his assistants may often simply forget to sterilize one of the instruments used to perform the surgical procedure, something that can be noticed at times when the surgery must continue anyway, so the surgeon uses his manual skills to proceed with only the instrument that has been prepared. In particular, this usually occurs with the irrigation and aspiration handpiece, so the surgeon needs to perform delicate movements only with the phaco handpiece.

Considering the above, the state of the art would clearly benefit from the introduction of a silicone sleeve used on the tip of the phaco handpiece, so that this surgical instrument can perform the same function as the irrigation and aspiration handpiece, thus making the use of the latter unnecessary.

### OBJECTIVES OF THE INVENTION

In order to solve the problems of the state of the art, it is an objective of the present invention to present the silicone sleeve specifically used on metallic tips of phaco handpieces used in phacoemulsification surgeries.

Another objective of the present application is to present the silicone sleeve with specific technical characteristics to allow the phaco handpiece to perform the same function of the irrigation and aspiration handpiece.

Finally, the objective of the present application is to make the use of the irrigation and aspiration handpiece unnecessary, in order to save resources and to make the preparation of this instrument for surgery unnecessary.

### SUMMARY OF THE INVENTION

In order to achieve the aforementioned objectives, the present invention comprises a solution that avoids all the aforementioned problems of the prior art. More specifically, the present invention comprises a sleeve with an inner wall that comprises threads to fit firmly onto the metallic tip of a phaco handpiece.

Said sleeve is made of flexible polymeric material, preferably silicone.

In particular, the sleeve of the present invention comprises a main body which, in turn, comprises a cylindrical base with a threaded section in its inner portion, said threaded section being distributed throughout the inner portion of the base. Furthermore, the base extends

### Superiorly to a Median Portion of Conical Shape.

In addition, the sleeve of the present invention further comprises a rod that is supported in the median portion and comprises a cylindrical shape, with a diameter considerably smaller than that of the elongated base, in which the rod also comprises a tip with a transverse and through-hole at its distal end. Furthermore, the rod comprises an internal passage channel that conforms an inner area and an upper through-hole, said through-hole being comprised vertically at the end of the rod.

The sleeve of the present invention is specifically fitted over an instrument used in surgical procedures to treat eye diseases such as cataracts. In particular, the sleeve is fitted over the tip of a phaco handpiece used in phacoemulsification surgeries, a procedure to remove a cataractous lens, and then replace that crystalline lens with an artificial lens in the patient's eye, wherein the procedure in question aims to facilitate the extraction of the crystalline lens while facilitating the patient's recovery.

To this end, the threaded section of the base is fitted over a corresponding threaded section of a phaco handpiece. After being fitted, the tip of the phaco handpiece, which is a metallic rod with one end curved like a hook, passes through the rod passage channel, until said curved end of the tip is fully covered by the rod.

The sleeve of the present invention, because it is made of flexible polymeric material, has a certain elasticity, so that it conforms to the tip of the phaco handpiece, and in particular fully covers said tip. Moreover, the hole in the rod corresponds to the aspiration aperture of the phaco handpiece, said aperture being responsible for aspirating fluids from inside the patient's eye during use of the phaco handpiece.

Additionally, the hole comprises a tapered upper portion to better control the aspiration flow of the softer components of the crystalline lens, in a delicate manner that does not present risks to the patient. In particular, this would be the function of an irrigation and aspiration handpiece, this handpiece being the other instrument used in phacoemulsification procedures, in which the hole, present on the sides of the rod, allows directing the irrigation flow of the Balanced Saline Solution (BSS) expelled by the phaco handpiece itself, in order to maintain the stability of the anterior chamber of the patient's eye, and also prevents possible burns from the incision made by surgical instruments by cooling their metallic tips.

Therefore, the sleeve of the present invention allows a phaco handpiece, usually used to remove the “harder” portions of a patient's crystalline lens, to perform more delicate movements, and remove the “softer” or less “hard” portions of the eye to complete phacoemulsification even without the irrigation and aspiration handpiece.

### BRIEF DESCRIPTION OF THE DRAWINGS

The subject matter of the present invention will become completely clear in its technical aspects from the detailed description that will be made based on the figures listed below, in which:

FIG. 1 shows a front view of the flexible sleeve for the tip of an ophthalmic surgical instrument;

FIG. 2 shows a front view of the flexible sleeve fitted over the tip of an ophthalmic surgical instrument;

FIG. 3 shows an approximate view of the flexible sleeve fitted over the tip of an ophthalmic surgical instrument;

FIG. 4 shows um top detail of the flexible sleeve fitted over the tip of an ophthalmic surgical instrument; and

FIG. 5 shows a photo of a surgical procedure that used an ophthalmic surgical instrument with the flexible sleeve.

### DETAILED DESCRIPTION

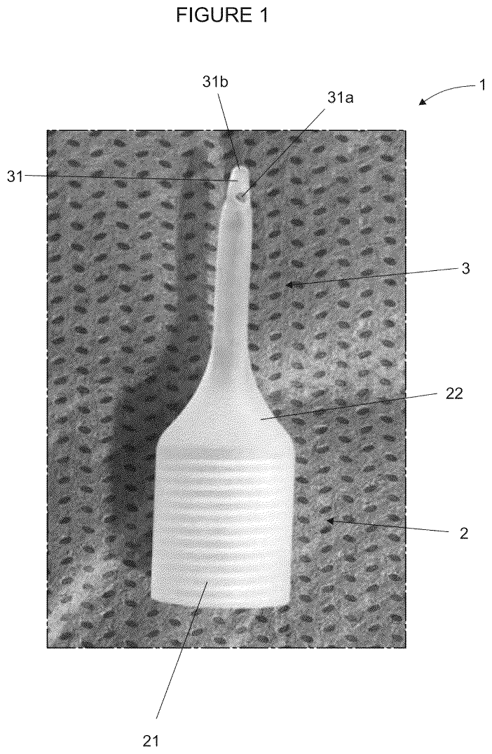

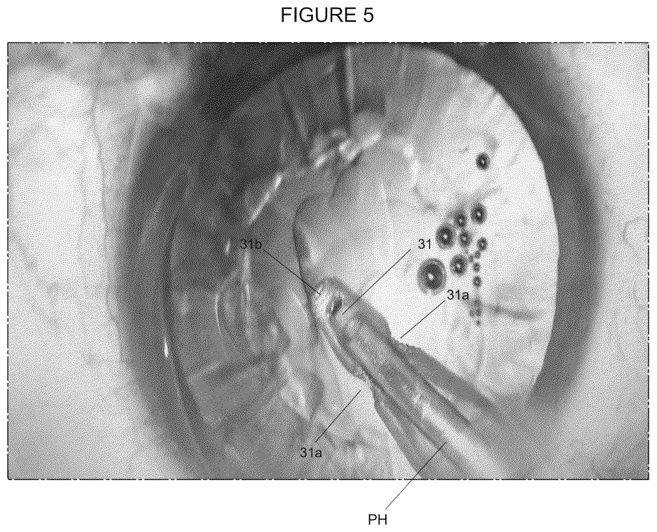

In accordance with the objectives mentioned and the figures presented, the present invention relates to a Flexible Sleeve for the Tip of an Ophthalmic Surgical Instrument, as illustrated by FIGS. 1 to 5, comprising a sleeve (1) made of flexible polymeric material, preferably silicone, and comprising a main body (2) and a central rod (3).

The main body (2) comprises a cylindrical base (21) with a threaded hole, said threaded hole being distributed throughout the inner portion of the base (21). Additionally, the base (21) extends longitudinally up to a conical shaped median portion (22).

The rod (3) extends from the median portion (22) and comprises a cylindrical shape with a diameter considerably smaller than the base (21), in which the rod (3) also comprises, at its distal end, a tip (31) with a transverse through-hole (31a). Moreover, the rod (3) comprises an internal passage channel that forms an inner area and an upper through-hole (31b), said through-hole (31b) being comprised longitudinally at the end of the rod (3).

The sleeve (1) of the present invention is specifically fitted over an instrument used in surgical procedures to treat eye diseases such as cataracts. In particular, the sleeve (1) is fitted over the tip of a phaco handpiece used in phacoemulsification surgeries, a procedure to remove a cataractous lens, and then replace that crystalline lens with an artificial lens in the patient's eye, wherein the procedure in question aims to facilitate the extraction of the lens while facilitating the patient's recovery.

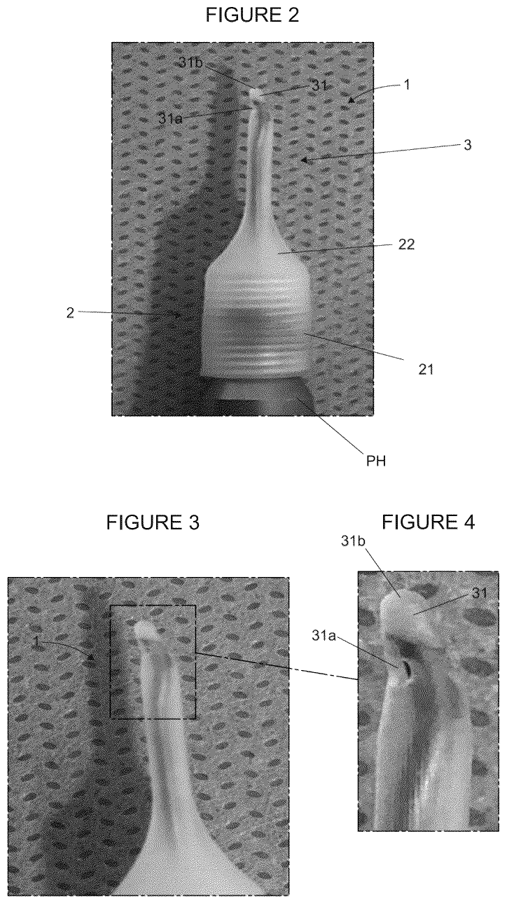

For this purpose, the threaded section of the base (2) is fitted into a corresponding threaded section of a phaco handpiece (PH). After being fitted, the tip of the phaco handpiece (PH), which, in the example shown, is a metallic rod with one end curved like a hook, said tip of the phaco handpiece (PH) passes through the rod passage channel (3), until said tip end is fully covered by the rod (3), as best illustrated in FIG. 2.

The sleeve (1) of the present invention, being made of flexible polymeric material, has a certain elasticity, so that it conforms to the tip of the phaco handpiece (PH), and in particular fully covers said tip. In addition, the hole (31b) of the rod (3) aligns with the aspiration aperture of the phaco handpiece (PH), said aperture being responsible for sucking fluids from inside the patient's eye after insertion of the tip of the phaco handpiece (PH).

The tip (31) comprises a dome to facilitate insertion into the patient's eye, in a delicate manner that does not present risks to the patient. Due to flexibility and elasticity of the sleeve (1) it is possible to aspirate more sensitive portions of the crystalline lens without causing damage to the patient's capsular bag, since, with the use of said sleeve (1), there are no abrupt aspirations of the tissue that can cause tears as those with the phaco handpiece (PH) without the use of the sleeve (1). In particular, this more delicate and fine aspiration would be the function of an irrigation and aspiration handpiece, this handpiece being the other instrument used in phacoemulsification procedures.

The holes (31a), present on the sides of the rod (3), as best illustrated in FIGS. 3 and 4, allow directing the irrigation flow of the Balanced Saline Solution (BSS) expelled by the phaco handpiece itself (PH), in order to maintain the stability of the anterior chamber of the patient's eye, and also prevents possible burns from the incision made by surgical instruments by cooling their metallic tips.

Therefore, the sleeve (1) of the present invention allows that a phaco handpiece (PH), usually used to remove the “harder” portions of a patient's eye, perform more delicate movements, and remove the “softer” or less “hard” portions of the eye to complete phacoemulsification even without the irrigation and aspiration handpiece. In other words, during the cataract removal procedure, the phaco handpiece (PH) is initially used, which emulsifies the cataract by means of ultrasound. So the initial removal of the hardest and thickest portions of the cataract is performed, still using the phaco handpiece (PH). Later, the phaco handpiece with the sleeve (1) is used for finer and more delicate aspiration where the irrigation and aspiration handpiece would usually be used.

More specifically, the sleeve (1) provides a similar tip shape of the irrigation and aspiration handpiece to the tip of the phaco handpiece (PH), with the aim of making the use of an irrigation and aspiration handpiece unnecessary. Thus, with the use of the sleeve (1), the irrigation and aspiration handpiece becomes unnecessary. Thereby, the purchase of equipment is no longer necessary, and the costs of maintenance and sterilization of such equipment are saved. With the decrease in costs, the surgery becomes more accessible, since, with the decrease in the cost of equipment to perform it, more surgeons will be able to perform such surgery.

Unless otherwise defined, all terms (including technical and scientific terms) used in this specification should be interpreted as is common in the prior art. Additionally, the features referred to herein in the singular may be one or more, unless the context clearly indicates otherwise. In the above descriptions, all terms of orientation, such as upper, lower, radial and axial, are used in relation to the drawings and should not be construed as limiting the invention, unless the context clearly indicates otherwise.

It is to be understood that the present description does not limit application to the details described herein and that the invention is capable of other embodiments and of being practiced or executed in a variety of ways, within the scope of the claims. Although specific terms have been used, such terms are to be construed in a generic and descriptive sense, and not for the purpose of limitation.

1. A FLEXIBLE SLEEVE FOR THE TIP OF AN OPHTHALMIC SURGICAL INSTRUMENT, the sleeve comprising a main body and a central rod wherein the main body comprises a cylindrical base with a threaded hole; the base extending longitudinally, in which at the opposite end to the base is the rod; said rod comprising an elongated cylindrical shape, in which the rod further comprises a tip with a transverse through-hole at its distal end, as well as an internal passage channel forming an inner area and a longitudinal through-hole; the sleeve being specifically fitted over the tip of a phaco handpiece (PH) used in phacoemulsification surgeries.

2. THE SLEEVE, according to claim 1, wherein the threaded section of the base is fitted into a corresponding threaded section of a phaco handpiece (PH), such that the tip of the phaco handpiece (PH) passes through the rod passage channel after being fitted, until said curved end of the tip is fully covered by the rod, the hole (31b) of the rod corresponding to the aspiration aperture of the phaco handpiece (PH).

3. THE SLEEVE, according to claim 1 wherein the sleeve {1} is made of a flexible polymeric material, preferably silicone, and conforms to the tip of the phaco handpiece (PH), fully covering said tip.

4. THE SLEEVE, according to claim 1 wherein the rod has a diameter considerably smaller than the base, and the through-hole is located vertically at the end of the rod
